(19) United States
(12) Patent Application Publication
(10) Pub. No.: US 2008/0292725 A1
(43) Pub. Date: Nov. 27, 2008

(54) COPPER-FREE FOOTBATH BLEND

(76) Inventors: Stephen P. Mixon, SR., Montgomery, TX (US); Rosemary Smithyman, Shreveport, LA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/123,763

(22) Filed: May 20, 2008

Related U.S. Application Data
(63) Continuation-in-part of application No. 12/016,718, filed on Jan. 18, 2008.
Application No. 12/016,718 is a continuation-in-part of application No. 11/750,223, filed on May 17, 2007.
Application No. 11/750,223 is a continuation-in-part of application No. 11/674,588, filed on Feb. 13, 2007.
Application No. 11/674,588 is a continuation of application No. 10/922,604, filed on Aug. 20, 2004, now Pat. No. 7,192,618.
(60) Provisional application No. 60/547,991, filed on Feb. 26, 2004.

(51) Int. Cl. A01N 59/16; A01P 1/00

(52) U.S. Cl. 424/642

(57) ABSTRACT

Provided is a composition suitable for treating a foot of an animal. The composition comprises zinc sulfate monohydrate, a chloride salt, and an acid, wherein the composition does not comprise copper. Also provided is a method for treating a foot of an animal. The method comprising contacting the foot of the animal with the above composition.

### CROSS-REFERENCE TO RELATED APPLICATIONS

This application is a continuation-in-part of U.S. application Ser. No. 12/016,718, filed on Jan. 18, 2008, which is a continuation-in-part of U.S. application Ser. No. 11/750,223, filed on May 17, 2007, which in turn is a continuation-in-part of U.S. application Ser. No. 11/674,588, filed on Feb. 13, 2007, which in turn is a Continuation of U.S. application Ser. No. 10/922,604, filed Aug. 20, 2004, now U.S. Pat. No. 7,192,618, which in turn claims priority to U.S. Provisional Application Ser. No. 60/547,991, filed Feb. 26, 2004; each of which are incorporated herein by reference in their entirety.

### FIELD

The present invention generally relates to compositions and methods for the treatment of a foot of an animal.

### BACKGROUND

Diseases of the foot of animals, particularly ungulates, present major health problems that can cause tremendous economic losses. Lameness in cows cause animals to lose weight and body conditioning. In dairy cows, milk production is lowered because the animals tend to go to feed less often. While diseases of the hoof are most prevalent in dairy cows, beef cattle, goats, sheep, and horses, other hoofed animals are all susceptible to these pathogens.

Common diseases of a foot of an animal, particularly the hooves of animals include hairy heel warts, foot rot, and foot scald.

Papillomatous digital dermatitis, also referred to as Hairy Heel Warts, Footwarts, and Strawberry Foot Disease, is an inflammation of the skin around the hoof of an animal. It is most commonly found at the back of the foot between the bulbs of the heels, but the disease can spread around the foot and into the cleft between the claws. Digital dermatitis is a very painful condition resulting in lameness, and subsequent reduced food intake and fertility. In dairy cows, animals infected with the disease also have reduced milk yields. Within a given herd, the proportion of animals affect by digital dermatitis can remain at low levels of less than 10% while in others, over half of the animals of a herd can be affected. The severity of the disease also varies from farm to farm.

The precise cause of digital dermatitis is not known for sure, but it is known that the disease is infectious in origin and that spiral bacteria are found in the lesions whereas they are not normally found in healthy skin. In fact, some strains of the disease are consistently found deep in damaged skin.

Foot rot or hoof rot, also known as infectious pododermatitis, is a hoof infection that is commonly found in cattle, sheep, and goats. The disease rots away the foot of the animal, more specifically the area between the two toes of the diseased animal. Like digital dermatitis, it is extremely painful and contagious.

In cattle, the cause of foot rot is thought to be two anaerobic bacteria, Fusobacterium necrophorum and Bacteroides melaninogenicus. Both bacteria are common to the environment that cattle live in and Fusobacterium is present in the rumen and fecal matter of the cattle. The disease is also caused by the microorganism Diechelobacter nodosus which can be found in contaminated soil. Another cause of foot rot may be high temperatures or humidity causing the skin between the hooves to crack, letting bacteria infect the foot. For this reason, foot rot is particularly prevalent in the southern region of the United States, where the south's wet and humid climate makes it conducive for foot rot and other diseases of the hoof.

Foot rot is also prevalent in sheep and goats and must be controlled. For instance, In a Virginia survey, approximately 21 percent of the producers considered foot rot to be a serious health problem in their flocks of sheep. As in cattle, foot rot in sheep and goats is extremely painful, contagious, and causes significant economic loss due to decreased flock production.

Foot scald, also referred to as benign foot rot or interdigital dermatitis, is an inflammation between the toes caused by F. necrophorum. Persistent moisture on the skin between the toes can increase susceptibility to foot scald. Foot scald often precedes foot rot. Like foot rot, foot scald outbreaks occur most often during persistent rainy weather along with high temperatures. If not treated these animals can become permanently infected. As with other infectious diseases of the hoof, foot scald causes stress to the animals and can affect weight gain, reproductive rates, and production. In addition, like other diseases of the hoof, foot scald incurs additional costs to the producer for treatment and increased labor during an outbreak.

Introducing an infected animal into a non-contaminated herd can create herd contamination and therefore all animals must be treated.

Some methods currently used to treat diseases of the hoof include antibiotic treatment, chemical treatment, and antimicrobial and disinfectants.

While antibiotics can be used to rapidly reduce the severity of diseases such as digital dermatitis and hoof rot, there are distinct disadvantages to using antibiotic therapy. For instance, the common and extensive use of antibiotics is likely to lead to the production of antibiotic resistant pathogens which are then not only problematic to the animals, but present a general concern to the public. In addition, treatment of herds with antibiotics are generally prohibitively expensive. Antibiotic therapy such as injections of long-acting penicillin or oxytetracycline, is effective, however, injections are costly and time consuming and residues may possibly be present in the milk. Although antibiotics such as oxytetracycline can be applied directly to a lesion, this becomes impractical for very large herds. While antibiotic powders can be used for treatment, and lincomycin/spectinomycin (LS-50) powder has proven to be successful, the powder is not readily absorbed into the hoof, and therefore may not reach all areas of infection. Furthermore, topical antibiotic agents can be rapidly deactivated by organic matter and are often toxic when ingested. Again, this method of administration is not optimal for the treatment of large herds.

Chemicals have also been used to treat some of these diseases. However, caustic chemicals themselves can cause serious lameness due to chemical burns on the skin, so therefore great care must be taken in their use. For example, formalin is OSHA regulated, carcinogenic, irritating to skin, less effective in cold temperature, and potentially leaves residue in milk.

Antimicrobial agents and disinfectants are often used in the treatment of infectious diseases of the hoof. Such products often include solutions of 10 percent copper sulfate or zinc sulfate. For example, copper sulfate crystals can be used directly but are becoming increasingly expensive, leave heavy copper residues in the footbath, can contaminate the soil with high levels of copper, and are difficult to measure and store.

Examples of antimicrobial compositions, and methods for preparing them, are described in U.S. Pat. Nos. 5,989,595; 6,242,011; 7,192,618, U.S. Patent Application Pub. No. 2005/0191365; 2005/0191394; 2005/0191395; 2006/0228308; 2007/0264398; 2007/0269563.

Footbaths currently used leave high concentrations of copper in both the soil and in the water. This is because most dairies dump the spent hoof baths into a manure pit or a lagoon so the copper ultimately gets spread on production ground with the manure. The practice can lead to copper accumulation in the soil. It is possible that, after several years, copper can accumulate in soil to levels that become toxic to soil microbes and crops. This can slow organic matter decomposition and nutrient cycling in soil and crop production could be reduced because of direct toxic effects of copper on the plants as well as reduced soil fertility. Importantly, copper accumulation in soil and forage can become toxic to sheep, whose tolerance for copper is much lower than that of dairy cattle. Toxic levels of copper in soil is a critical issue because there is no practical way to reverse the problem.

Accordingly, there is a need for compositions and methods for the treatment of infectious diseases of the feet of animals which not only exert potent antimicrobial properties, but also provide for reduction in the levels of copper required.

### SUMMARY

The present application provides an effective footbath blend that lacks copper.

Thus, in some embodiments, the application is directed to a composition suitable for treating a foot of an animal. The composition comprises zinc sulfate monohydrate, a chloride salt, and an acid, where the composition does not comprise copper.

The application is also directed to a method for treating a foot of an animal. The method comprises contacting the foot of the animal with the above-described composition.

### DETAILED DESCRIPTION

In accordance with the present application, it has been discovered that compositions described herein can be used to effectively control diseases of the hoof, such as hairy heel foot warts, foot rot, foot scald, or other infectious hoof conditions, without the use of copper. Generally, the compositions for use in the methods described herein contain FDA GRAS listed ingredients and are non-toxic, safe, stable, and easy to use. Also, the methods and compositions for treatment of hoof disease described herein do not require usage of antibiotics or formaldehyde. Furthermore, the methods described herein eliminate copper deposits in soil and lagoons, unlike other available treatments.

The present application is generally directed to compositions and methods for the treatment of infectious diseases of a foot of an animal, particularly the foot/hoof of an ungulate animal. The antimicrobial composition can be used, for example, in a concentrated form, a diluted form, or as a powder.

In various embodiments, the antimicrobial compositions are bacteriostatic agents that are non-toxic and composed of FDA GRAS materials. The disinfection compositions and methods of the present invention have activity against a wide variety of microorganisms such as Gram positive and Gram negative bacteria, yeast, molds, bacterial spores, viruses, etc. The antimicrobial composition can provide antimicrobial effects against microbes including, but not limited to, Fusobacterium necrophorum, Bacteroides melaninogenicus, Dichelobacter nodosus, Staphylococcus intermedius, Streptococcus mutans, Escherichia coli, Staphylococcus aureus, Listeria monocytogenes, Salmonella typhimurium, Salmonella (nontyphoidal), Pseudomonas, Vibrio parahaemolyticus, V. vulnificus, V. mimicus, V. fluvialis, V. furnissii, V. hollisae, V. cholerae, (and other Vibrio spp.), Bacillus cereus, Clostridium botulinum, Clostridium perfringens, Campylobacter jejuni, Shigella, and the like. The methods described herein particularly benefit from the composition's antimicrobial effects against Fusobacterium necrophorum, Bacteroides melaninogenicus, and Diechelobacter nodosus.

Another realized benefit of the antimicrobial composition is the maintenance of low levels of digital dermatitis (i.e., heel warts) and interdigital dermatitis (i.e., heel erosion and/or foot rot) with routine use. The antimicrobial composition of the invention, when used in a regular footbath program, helps to promote hoof health. Healthy hooves translate to a reduction in hoof diseases and maintenance of a low incidence of these diseases in the herd. This in turn can prevent the tremendous economic losses discussed above, which can occur when animals are afflicted with these diseases.

In addition to the efficacy of antimicrobial compositions, environmental benefits can be appreciated with the present invention due to the elimination of copper used in the footbaths.

Thus, the present application is directed to a composition suitable for treating a foot of an animal. The composition comprises zinc sulfate monohydrate, a chloride salt, and an acid; the composition does not comprise copper.

The compositions are not narrowly limited to any particular chloride salt. Examples of useful such salts include aluminum, ammonium, barium, calcium, ferric, magnesium, manganese, mercuric, nickel, potassium, sodium, stannic, tin, and zinc chloride salts. Preferably, the chloride salt of these compositions is sodium chloride or potassium chloride, most preferably sodium chloride.

Exemplary acids for the compositions include those provided in Table 1. In preferred embodiments, the acid is sodium bisulfate, sodium acid pyrophosphate, citric acid, lactic acid, or sulfuric acid. More preferably, the acid is sodium bisulfate or sodium acid pyrophosphate. Most preferably, the acid is sodium acid pyrophosphate.

TABLE 1
Acids Generally Recognized as Safe (GRAS)

| Acid Name | CAS No. |
|---|---|
| ACETIC ACID | 000064-19-7 |
| ACONITIC ACID | 000499-12-7 |
| ADIPIC ACID | 000124-04-9 |
| ALGINIC ACID | 009005-32-7 |
| P-AMINOBENZOIC ACID | 000150-13-0 |
| AMINO TRI(METHYLENE PHOSPHONIC ACID), SODIUM SALT | 020592-85-2 |
| ANISIC ACID | 001335-08-6 |
| ASCORBIC ACID | 000050-81-7 |
| L-ASPARTIC ACID | 000056-84-8 |
| BENZOIC ACID | 000065-85-0 |
| N-BENZOYLANTHRANILIC ACID | 000579-93-1 |
| BORIC ACID | 010043-35-3 |
| (E)-2-BUTENOIC ACID | 003724-65-0 |
| BUTYRIC ACID | 000107-92-6 |
| CHOLIC ACID | 000081-25-4 |
| CINNAMIC ACID | 000621-82-9 |
| CITRIC ACID | 000077-92-9 |
| CYCLOHEXANEACETIC ACID | 005292-21-7 |
| CYCLOHEXANECARBOXYLIC ACID | 000098-89-5 |
| DECANOIC ACID | 000334-48-5 |
| 5-DECENOIC ACID | 085392-03-6 |
| 6-DECENOIC ACID | 085392-04-7 |
| 9-DECENOIC ACID | 014436-32-9 |
| (E)-2-DECENOIC ACID | 000334-49-6 |
| 4-DECENOIC ACID | 026303-90-2 |
| DEHYDROACETIC ACID | 000520-45-6 |
| DESOXYCHOLIC ACID | 000083-44-3 |
| 2,4-DIHYDROXYBENZOIC ACID | 000089-86-1 |
| 3,7-DIMETHYL-6-OCTENOIC ACID | 000502-47-6 |
| 2,4-DIMETHYL-2-PENTENOIC ACID | 066634-97-7 |
| ERYTHORBIC ACID | 000089-65-6 |
| 2-ETHYLBUTYRIC ACID | 000088-09-5 |
| 4-ETHYLOCTANOIC ACID | 016493-80-4 |
| FOLIC ACID | 000059-30-3 |
| FORMIC ACID | 000064-18-6 |
| FUMARIC ACID | 000110-17-8 |
| GERANIC ACID | 000459-80-3 |
| GIBBERELLIC ACID | 977136-81-4 |
| D-GLUCONIC ACID | 000526-95-4 |
| L-GLUTAMIC ACID | 000056-86-0 |
| GLUTAMIC ACID HYDROCHLORIDE | 000138-15-8 |
| GLYCOCHOLIC ACID | 000475-31-0 |
| HEPTANOIC ACID | 000111-14-8 |
| (E)-2-HEPTENOIC ACID | 018999-28-5 |
| HEXANOIC ACID | 000142-62-1 |
| TRANS-2-HEXENOIC ACID | 013419-69-7 |
| 3-HEXENOIC ACID | 004219-24-3 |
| HYDROCHLORIC ACID | 007647-01-0 |
| 4-HYDROXYBENZOIC ACID | 000099-96-7 |
| 4-HYDROXYBUTANOIC ACID LACTONE | 000096-48-0 |
| 4-HYDROXY-2-BUTENOIC ACID GAMMA-LACTONE | 000497-23-4 |
| 5-HYDROXY-2,4-DECADIENOIC ACID DELTA-LACTONE | 027593-23-3 |
| 5-HYDROXY-2-DECENOIC ACID DELTA-LACTONE | 051154-96-2 |
| 5-HYDROXY-7-DECENOIC ACID DELTA-LACTONE | 025524-95-2 |
| 4-HYDROXY-2,3-DIMETHYL-2,4-NONADIENOIC ACID GAMMA LACTONE | 000774-64-1 |
| 6-HYDROXY-3,7-DIMETHYLOCTANOIC ACID LACTONE | 000499-54-7 |
| (Z)-4-HYDROXY-6-DODECENOIC ACID LACTONE | 018679-18-0 |
| 5-HYDROXY-2-DODECENOIC ACID LACTONE | 016400-72-9 |
| 1-HYDROXYETHYLIDENE-1,1-DIPHOSPHONIC ACID | 002809-21-4 |
| 2-(2-HYDROXY-4-METHYL-3-CYCLOHEXENYL)PROPIONIC ACID GAMMA- | 057743-63-2 |
| LACTONE | |
| 4-HYDROXY-4-METHYL-7-CIS-DECANOIC ACID GAMMALACTONE | 070851-61-5 |
| 5-HYDROXY-4-METHYLHEXANOIC ACID DELTA-LACTONE | 010413-18-0 |
| 4-HYDROXY-4-METHYL-5-HEXENOIC ACID GAMMA LACTONE | 001073-11-6 |
| 4-HYDROXY-3-METHYLOCTANOIC ACID LACTONE | 039212-23-2 |
| HYDROXYNONANOIC ACID, DELTA-LACTONE | 003301-94-8 |
| 3-HYDROXY-2-OXOPROPIONIC ACID | 001113-60-6 |
| 4-HYDROXY-3-PENTENOIC ACID LACTONE | 000591-12-8 |
| 5-HYDROXYUNDECANOIC ACID LACTONE | 000710-04-3 |
| 5-HYDROXY-8-UNDECENOIC ACID DELTA-LACTONE | 068959-28-4 |
| ISOBUTYRIC ACID | 000079-31-2 |
| ISOVALERIC ACID | 000503-74-2 |
| ALPHA-KETOBUTYRIC ACID | 000600-18-0 |
| LACTIC ACID | 000050-21-5 |
| LAURIC ACID | 000143-07-7 |
| LEVULINIC ACID | 000123-76-2 |
| LIGNOSULFONIC ACID | 008062-15-5 |
| LINOLEIC ACID | 000060-33-3 |
| L-MALIC ACID | 000097-67-6 |
| MALIC ACID | 000617-48-1 |
| 2-MERCAPTOPROPIONIC ACID | 000079-42-5 |
| 2-METHOXYBENZOIC ACID | 000579-75-9 |
| 3-METHOXYBENZOIC ACID | 000586-38-9 |
| 4-METHOXYBENZOIC ACID | 000100-09-4 |
| TRANS-2-METHYL-2-BUTENOIC ACID | 000080-59-1 |
| 2-METHYLBUTYRIC ACID | 000116-53-0 |
| 3-METHYLCROTONIC ACID | 000541-47-9 |
| 2-METHYLHEPTANOIC ACID | 001188-02-9 |
| 2-METHYLHEXANOIC ACID | 004536-23-6 |
| 5-METHYLHEXANOIC ACID | 000628-46-6 |
| 4-METHYLNONANOIC ACID | 045019-28-1 |
| 4-METHYLOCTANOIC ACID | 054947-74-9 |
| 3-METHYL-2-OXOBUTANOIC ACID | 000759-05-7 |
| 3-METHYL-2-OXOPENTANOIC ACID | 001460-34-0 |
| 4-METHYL-2-OXOPENTANOIC ACID | 000816-66-0 |
| 3-METHYLPENTANOIC ACID | 000105-43-1 |
| 4-METHYLPENTANOIC ACID | 000646-07-1 |
| 2-METHYL-2-PENTENOIC ACID | 003142-72-1 |
| 2-METHYL-3-PENTENOIC ACID | 037674-63-8 |
| 2-METHYL-4-PENTENOIC ACID | 001575-74-2 |
| 4-METHYLPENT-2-ENOIC ACID | 010321-71-8 |
| 3-METHYL-3-PHENYL GLYCIDIC ACID ETHYL ESTER | 000077-83-8 |
| 4-(METHYLTHIO)-2-OXOBUTANOIC ACID | 000583-92-6 |
| 2-METHYLVALERIC ACID | 000097-61-0 |
| MYRISTIC ACID | 000544-63-8 |
| NITRIC ACID | |
| NONANOIC ACID | 000112-05-0 |
| (E)-2-NONENOIC ACID | 014812-03-4 |
| 2-NONENOIC ACID GAMMA-LACTONE | 021963-26-8 |
| 9,12-OCTADECADIENOIC ACID (48%) AND 9,12,15-OCTADECATRIENOIC | 977043-76-7 |
| ACID (52%) | |
| OCTANOIC ACID | 000124-07-2 |
| (E)-2-OCTENOIC ACID | 001871-67-6 |
| OLEIC ACID | 000112-80-1 |
| 3-OXODECANOIC ACID GLYCERIDE | 128331-45-3 |
| 3-OXODODECANOIC ACID GLYCERIDE | 128362-26-5 |
| 3-OXOHEXADECANOIC ACID GLYCERIDE | 128331-46-4 |
| 3-OXOHEXANOIC ACID DIGLYCERIDE | 977148-06-3 |
| 3-OXOOCTANOIC ACID GLYCERIDE | 128331-48-6 |
| 2-OXOPENTANEDIOIC ACID | 000328-50-7 |
| 2-OXO-3-PHENYLPROPIONIC ACID | 000156-06-9 |
| 3-OXOTETRADECANOIC ACID GLYCERIDE | 128331-49-7 |
| PALMITIC ACID | 000057-10-3 |
| 4-PENTENOIC ACID | 000591-80-0 |
| 2-PENTENOIC ACID | 013991-37-2 |
| PERACETIC ACID | 000079-21-0 |
| PERIODIC ACID | 010450-60-9 |
| PHENOXYACETIC ACID | 000122-59-8 |
| PHENYLACETIC ACID | 000103-82-2 |
| 3-PHENYLPROPIONIC ACID | 000501-52-0 |
| PHOSPHORIC ACID | 007664-38-2 |
| POLY(ACRYLIC ACID-CO-HYPOPHOSPHITE), SODIUM SALT | 071050-62-9 |
| POLYACRYLIC ACID, SODIUM SALT | 009003-04-7 |
| POLYMALEIC ACID | 026099-09-2 |
| POLYMALEIC ACID, SODIUM SALT | 030915-61-8 |
| POTASSIUM ACID PYROPHOSPHATE | 014691-84-0 |
| POTASSIUM ACID TARTRATE | 000868-14-4 |
| PROPIONIC ACID | 000079-09-4 |
| 2-(4-METHYL-2-HYDROXYPHENYL)PROPIONIC ACID-GAMMA-LACTONE | 065817-24-5 |
| PYROLIGNEOUS ACID | 008030-97-5 |
| PYRUVIC ACID | 000127-17-3 |
| SALICYLIC ACID | 000069-72-7 |
| SODIUM ACID PYROPHOSPHATE | 007758-16-9 |
| SODIUM BISULFATE (SODIUM ACID SULFATE) | |
| SORBIC ACID | 000110-44-1 |
| STEARIC ACID | 000057-11-4 |
| SUCCINIC ACID | 000110-15-6 |
| SULFAMIC ACID | 005329-14-6 |
| SULFURIC ACID | 007664-93-9 |
| SULFUROUS ACID | 007782-99-2 |
| TANNIC ACID | 001401-55-4 |
| TARTARIC ACID, L | 000087-69-4 |
| TAUROCHOLIC ACID | 000081-24-3 |
| 1,2,5,6-TETRAHYDROCUMINIC ACID | 056424-87-4 |
| THIOACETIC ACID | 000507-09-5 |
| THIODIPROPIONIC ACID | 000111-17-1 |
| TRIFLUOROMETHANE SULFONIC ACID | 001493-13-6 |
| (2,6,6-TRIMETHYL-2-HYDROXYCYCLOHEXYLIDENE)ACETIC ACID | 015356-74-8 |
| GAMMA-LACTONE | |
| UNDECANOIC ACID | 000112-37-8 |
| 10-UNDECENOIC ACID | 000112-38-9 |
| N-UNDECYLBENZENESULFONIC ACID | 050854-94-9 |
| VALERIC ACID | 000109-52-4 |
| VANILLIC ACID | 000121-34-6 |

In some embodiments, the composition further comprises a second acid. The second acid can also be any acid but is preferably sodium bisulfate, sodium acid pyrophosphate, citric acid, lactic acid, or sulfuric acid, more preferably sodium bisulfate or sodium acid pyrophosphate. When the first acid is sodium acid pyrophosphate, the second acid is preferably sodium bisulfate or citric acid, most preferably sodium bisulfate. The composition can also comprise a third, fourth, etc. acid.

The antimicrobial compositions of this application also contain an antimicrobial metal-containing agent capable of providing free metal ions in solution (see e.g., U.S. Patent App. Pub. No. 2005/0191365, incorporated herein by reference). In some embodiments, the antimicrobial metal of the composition is zinc, magnesium, silver, and/or iron. Preferably, the antimicrobial metal is zinc. The antimicrobial composition can contain at least one antimicrobial metal, at least two antimicrobial metals, at least three antimicrobial metals, or at least four antimicrobial metals.

In some embodiments, the antimicrobial composition contains zinc at a concentration of at least about 1 ppm during use (i.e., after diluting a concentrate or powder to its recommended concentration for treatment—see below). In some embodiments, the diluted antimicrobial composition contains about 1 ppm to about 300 ppm zinc. For example, the diluted antimicrobial composition can contain about 10 ppm to about 150 ppm zinc. Preferably, the diluted antimicrobial composition contains about 15 ppm to about 75 ppm zinc. Zinc can be supplied in the antimicrobial compositions described herein in a variety of forms such as zinc sulfate, preferably, zinc sulfate monohydrate.

In these compositions, the zinc sulfate monohydrate is preferably about 10-80% of the composition, the chloride salt is preferably about 15-60% of the composition, and the acid and second acid (if present) is preferably about 0.5-5% of the composition, by weight. More preferably, the zinc sulfate monohydrate is about 40-75% of the composition, the chloride salt is about 20-55% of the composition, and the acid and second acid is about 1-5% of the composition, by weight.

In even more preferred embodiments, the zinc sulfate monohydrate is about 70% of the composition, the sodium chloride is about 27% of the composition, and the acid and second acid is about 3% of the composition, by weight. Preferably here, the acid and second acid is sodium acid pyrophosphate and sodium bisulfate, wherein the sodium acid pyrophosphate is about 2% of the composition and the sodium bisulfate is about 1% of the composition.

Zinc can be supplied in the antimicrobial compositions described herein in a variety of forms such as zinc sulfate. Preferably, the zinc is supplied as zinc sulfate monohydrate.

An antimicrobial composition can be used at a range of effective concentrations. In some embodiments, an antimicrobial composition has a pH of about 0.5 to about 5.0. In other embodiments, an antimicrobial composition has a pH of about 1.0, about 1.5, about 2.0, about 2.5, about 3.0, about 3.5, about 4.0, or about 4.5. Preferably, an aqueous antimicrobial composition has a pH of about 1.5 to 2.0 upon initial treatment; as treatment progresses, the pH can increase through at least about 4.5 and remain effective. Preferably, a foam or gel antimicrobial composition has a pH of about 0.5 to about 1.5, more preferably, about 1.0. In some embodiments, an antimicrobial composition comprises an aqueous solution of water, sulfuric acid, and sodium sulfate and/or copper sulfate, and the antimicrobial composition has a pH of about 1.0 to about 4.5. The antimicrobial composition can be modified for increased buffering so as to further increase the pH at which the composition retains effectiveness. Various additives to increase buffering capacity and extend effective pH range are as described in U.S. Prov. App. Ser. No. 60/982,494, incorporated herein by reference in its entirety. As an example, the concentration of zinc can be increased so as to extend the effective pH range of an antimicrobial composition.

The antimicrobial composition can be a blended composition of ingredients described herein, thus forming a homogenous mixture of the solids. The antimicrobial composition can be packaged in dry blended form (e.g., packaged in about 10 lb—about 50 lb bags). The dry blended form can be directly applied to the foot of an animal as a powder or dust. The dry blended form can also be mixed with water to form a concentrated antimicrobial composition. The dry blended form is preferably mixed with water to form a diluted composition.

In various embodiments, the present invention can also involve kits for use in the treatment of an infectious disease of a foot of an animal. Such kits can include the compositions of the present invention and, in certain embodiments, instructions for administration and/or usage. When supplied as a kit, the different components of the composition can be packaged in separate containers and admixed immediately before or during use. Such packaging of the components separately can, if desired, be presented in a pack or dispenser device which may contain one or more unit dosage forms containing the composition. Such packaging of the components separately can also, in certain instances, permit long-term storage without losing activity of the components. In various embodiments, the different components can be packaged in one composition for administration together.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, thumb disc, videotape, audio tape, and the like. Detailed instructions may not be physically associated with the kit; instead, a user may be directed to an Internet web site specified by the manufacturer or distributor of the kit.

The compositions of the application are preferably diluted, most preferably in water, to prepare for use. Preferably, the composition is diluted between about 80-fold to about 400-fold in water. More preferably, the composition is diluted between about 80-fold to about 400-fold in water. Most preferably, the composition is diluted about 250-fold in water.

Another aspect of the invention is directed towards treatment of a foot of an animal by administration of one or more of the compositions described herein. Infectious diseases of the foot of an animal that can be treated or prevented with compositions and methods described herein include, but are not limited to hairy heel warts, foot rot, and/or scald or other conditions caused by Fusobacterium necrophorum, Bacteroides melaninogenicus, and/or Diechelobacter nodosus. As used herein, treatment is generally understood to encompass both prophylactic treatment as well as treatment of an existing or diagnosed condition. For example, an animal in need of treatment can be at risk, or determined to be at risk, for an infection of the foot. As another example, an animal in need of treatment can have, or be diagnosed as having, an infection of the foot. Diagnosis and risk assessment for animal foot diseases discussed herein is within the skill of the art. In some cases (e.g., with hoof rot), the treatment is preferably to increase hoof hardness, thus helping to prevent the infection.

In some embodiments, the method for treating a foot of an animal comprises contacting a foot of an animal with any of the above-identified compositions. Preferably, the composition is diluted between about 10-fold to about 200-fold in water. In more preferred embodiments, the composition before dilution comprises zinc sulfate monohydrate at about 70% of the composition, sodium chloride at about 27% of the composition, sodium acid pyrophosphate at about 2% of the composition, and sodium bisulfate at about 1% of the composition. Most preferably, that composition is diluted about 250-fold in water before treating the foot of the animal.

In various embodiments, the animal to be treated is an ungulate. An ungulate is understood to include an animal having hooves, or feet resembling hooves, or feet that are hoof-like. An ungulate is also understood to include an animal of, or belonging to, the former order Ungulata, now divided into the orders Perissodactyla and Artiodactyla and composed of hoofed mammals such as, but not limited to, a horse, a cow, a goat, a sheep, a pig, deer, an elephant, an elk, a bison, a moose, a gazelle, and an antelope. Preferably, the subject of various treatments described herein is a cow, a sheep, a horse, or a goat. More preferably, the ungulate to be treated with an antimicrobial composition of the present invention is a cow. Infectious diseases in the hooves of cows are most prevalent in dairy cow herds but are also problematic in beef cattle. The treatment compositions and methodologies described herein can be directed to dairy and/or beef cattle. In some embodiments, the ungulate to be treated with a composition of the present invention is a goat. Research has shown that some goats (e.g., Boer goats) are more prone to foot rot than others. In some embodiments, the ungulate to be treated with an antimicrobial composition of the present invention is a horse. In other embodiments, the ungulate to be treated with an antimicrobial composition of the present invention is a sheep. Research has also shown that some breeds of sheep (e.g., Merino sheep) are more susceptible to foot rot and foot scald than other breeds.

An animal foot can be contacted with a composition described herein by any method or apparatus suitable for applying the antimicrobial agent. For example, an antimicrobial composition can be administered by a method such as a footbath, powder, spray, gel, foam, or a combination thereof. Various embodiments provides for components of the footbath, spray, gel or foam form of the composition to be administered sequentially. In other embodiments of the present invention, a footbath, spray, gel or foam form of an antimicrobial composition can be administered sequentially with an antimicrobial composition in the same or another form such as a footbath, powder, spray, gel, or foam.

Preferably, the composition is administered as a footbath. An animal's foot can be exposed to a footbath containing an antimicrobial composition described herein by a variety of means known to the art, which include, for example, a trough. Another option application via footbath is the use of absorptive pads saturated with the composition to be used for treatment. The pad can be placed in a high traffic area that an animal must pass through, such as a gate, lane, or water trough. When an animal stands to drink at the water, feet can be treated.

In various embodiments, an antimicrobial composition described herein can be administered as a spray. An antimicrobial composition can be applied using fixed or articulating nozzles, at higher pressures, varying or steady flow rates, various temperatures, and/or with or without agitation or brushes. Spraying can be accomplished by an apparatus such as a spray cabinet with stationary or moving spray nozzles. The nozzles can create a mist, vapor, or spray that contacts an animal's feet. The spray can be set up as a walk-through pen or in a holding pen.

Application of a material by spray can be accomplished, for example, using a manual spray wand application, an automatic spray of the animals moving through a gate or room or gateway, or the like. Multiple spray heads to ensure complete contact or other spray means may be used. One preferred automatic spray application involves the use of a spray booth. The spray booth substantially confines the sprayed composition to within the parameter of the booth. The spray booth can include steam jets that can be used to apply an antimicrobial compositions of the invention. The spray pattern can be virtually any useful spray pattern.

In additional embodiments, the composition can be administered as a foam. The foam can be prepared, for example, by mixing foaming surfactants with the antimicrobial composition. The foaming surfactants can be nonionic, anionic, or cationic in nature. Examples of useful surfactant types include, but are not limited to the following: alcohol ethoxylates, alcohol ethoxylate carboxylate, amine oxides, alkyl sulfates, alkyl ether sulfate, sulfonates, quaternary ammonium compounds, alkyl sarcosines, betaines, and alkyl amides. The foaming surfactant is typically mixed at time of use with the antimicrobial composition but can be prepared in advance of the time of use. At time of use, compressed air can be injected into the mixture, and the foam can be applied to a foot of an animal. Thickeners (e.g., xanthan gum, polymeric thickeners, cellulose thickeners, propylene glycol, glycerin, or the like) can be further combined to produce a foam which may remain in contact with the infected area of the foot for a longer period of time than a formulation without the thickener. In some embodiments, a foam antimicrobial composition contains about 5 wt % to about 20 wt % of thickeners. As an example, a foam antimicrobial composition can contain about 5 wt % to about 10 wt % propylene glycol and about 5 wt % to about 10 wt % glycerin.

In further embodiments, the composition can be administered as a gel. The animal foot can be treated with a thickened or gelled version of an antimicrobial composition. In the thickened or gelled state, the antimicrobial composition can remain in contact with the animal's foot for longer periods of time, thus increasing the antimicrobial efficacy. The thickened or gelled solution can also better adhere to vertical surfaces and crevices in the animal's foot. The composition can be thickened or gelled using thickening agents including, but not limited to, xanthan gum, polymeric thickeners, cellulose thickeners, propylene glycol, glycerin, or the like. Rod micelle forming systems such as amine oxides and anionic counter ions could also be used. The thickeners or gel forming agents can be used, for example, in the concentrated product or by mixing with the antimicrobial composition at time of use. Exemplary use levels of thickeners or gel agents can range from about 100 ppm to about 10%, by weight.

Immersing an animal's foot in a liquid or powder disinfectant, of the present invention, can be accomplished by any of a variety of methods known to those of skill in the art. For example, troughs can be used to immerse the feet. A disinfectant composition of the present invention, contained in the trough, can be agitated so as to increase the application and/or absorption of the solution into or onto the feet. Agitation can be obtained by conventional methods, including ultrasonics, aeration by bubbling air through the solution, by mechanical methods, such as strainers, paddles, brushes, pump driven liquid jets, or by combinations of these methods. The disinfection agent can be heated to increase the efficacy of the solution in killing microorganisms. After the foot has been immersed for a time sufficient for the desired effect, the foot can be removed from the bath and the antimicrobial composition can be rinsed, drained, blotted, or evaporated from the foot. Treatments with any of the compositions of the present invention can be applied one time or repeatedly within a short period of time (minutes or hours), or the treatments can be repeated as needed over a longer period of time (days or months).

The antimicrobial compositions described herein can be contacted with an animal foot/hoof in an amount effective to result in a reduction significantly greater than is achieved by washing with water, or at least a 50% reduction, preferably at least a 90% reduction, more preferably at least a 99% reduction, in the resident microbial preparation.

The present methods usually require a certain minimal contact time of the composition with the foot/hoof of an animal for occurrence of significant disinfection effect. The contact time can vary with concentration of the use composition, method of applying the use composition, temperature of the use composition, amount of soil and/or contamination on the hoof and/or in the footbath, number of microorganisms present on the hoof, type and formulation of the disinfection composition, or the like. The minimum exposure time is, for example, at least about 2 to about 5 seconds. The exposure time can be, for example, at least about 5 seconds, at least about 10 seconds, at least about 15 seconds, at least about 30 seconds, at least about 45 seconds, at least about one minute, at least about two minutes, at least about three minutes, at least about four minutes, at least about five minutes, at least about six minutes, at least about seven minutes, at least about eight minutes, at least about nine minutes, at least about ten minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes. Even longer exposure times are contemplated, for example, several hours or even days. Generally, longer exposure times to the disinfection composition will be preferred as the pH increases and/or the antimicrobial metal (e.g., zinc) content decreases. After direct exposure during administration, it is contemplated that the disinfection composition can remain in contact with a hoof for extended periods of time.

Application of the composition to an animal's foot can occur in periodic sequential applications. An effective amount of the composition can be applied to an animal's foot several times per day and/or several times over a period of several days. For example, the disinfection composition can be applied to part or all of an animal's foot/hoof about every one hour, about every two hours, about every three hours, about every four hours, about every five hours, about every six hours, about every seven hours, about every eight hours, about every nine hours, about every ten hours, about every eleven hours, or about every twelve hours. Longer periods of time between applications are contemplated. For example, the antimicrobial composition can be applied every day, every other day, every three days, or every several days.

In some embodiments, the animal has, or is at risk for, an infectious disease of the foot. As an example, an animal can be in need of treatment with the compositions described herein when the animal is diagnosed with hairy heel warts, foot rot, and/or scald. As another example, an animal can be in need of treatment with the compositions described herein when the animal is determined at risk for hairy heel warts, foot rot, and/or scald. Diagnosis of infectious diseases of the foot in animals is within the skill of the art. The determination of risk for an infectious disease of the foot can be according to environmental conditions, susceptibility of certain types of animals, or other factors known to one of skill in the art.

The following non-limiting example is provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

### Copper-Free Footbath Blend On-Farm Trials

Objective: Evaluate the efficacy of Tasker Products™ Copper-Free Footbath Blend (dry) comparable to copper sulfate and/or zinc sulfate based footbath products and topical solutions.

Trial Sites: 4 commercial diary farms ranging in size from 200-1,200 cows across 3 states (Texas, Louisiana and Connecticut) to provide representation of US dairy market within different geographies.

Formulation/Preparation for Treatment:

The Copper-Free Footbath Blend used was of the following formulation:

70% zinc sulfate monohydrate

27% sodium chloride

2% sodium acid pyrophosphate

1% sodium bisulfate.

0.3 lb. of the above formulation was diluted in 1 gal. water (generally prepared as 15 lb/50 gal. water), a dilution of about 27.8-fold.

Treatment: The animals were treated using the above footbath formulation by the following schedule:

Once per day for 35 days, then five days a week for 7 weeks.

Methods of Assessment: Footbath Application

Hoof Scoring (2 Farms)
Pain score assessment and direct observation/evaluation
   Rear feet
   Full milking string
Lesions identified and scored
   Number
   Size
   Stage/Severity
Assessment timelines
   Pre-trial baseline
   Mid term (3 weeks)
   End of trial (6 weeks)

Hoof Trimmer Report (2 Farms)
Hoof hardness/hoof condition assessed and reported
PDD levels recorded
   # lesions wrapped as a percentage of cows trimmed
Assessment timelines
   Pre-trial baseline
   Mid term (3 weeks)
   End of trial (6 weeks)

Results:

Hoof Trimmer Reports Indicate
Improvement in overall hoof condition (1 dairy)
Maintenance of good hoof condition (1 dairy)
Reduction in incidence of heel warts (1 dairy)
Maintenance of low heel wart and footrot levels (1 dairy)

Hoof Scoring Assessment Indicates
Reductions in pain scores (2 dairies) indicating reduced lesion severity
Indications of reduction in lesion activity (drying, darkening, shrinking of lesion margins).

Owner reports of improved locomotion (reduced lameness)—2 dairies

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A composition suitable for treating a foot of an animal, the composition comprising zinc sulfate monohydrate, a chloride salt, and an acid, wherein the composition does not comprise copper.

2. The composition of claim 1, wherein the chloride salt is sodium chloride or potassium chloride.

3. The composition of claim 1, wherein the acid is sodium bisulfate, sodium acid pyrophosphate, citric acid, lactic acid, or sulfuric acid.

4. The composition of claim 1, wherein the acid is sodium bisulfate or sodium acid pyrophosphate.

5. The composition of claim 1, wherein the acid is sodium acid pyrophosphate.

6. The composition of claim 5, further comprising a second acid, wherein the second acid is sodium bisulfate or citric acid.

7. The composition of claim 6, wherein the zinc sulfate monohydrate is about 10-80% of the composition, the chloride salt is about 15-60% of the composition, and the acid and second acid is about 0.5-5% of the composition, by weight.

8. The composition of claim 7, wherein the zinc sulfate monohydrate is about 40-75% of the composition, the chloride salt is about 20-55% of the composition, and the acid and second acid is about 1-5% of the composition, by weight.

9. The composition of claim 7, wherein the zinc sulfate monohydrate is about 70% of the composition, the sodium chloride is about 27% of the composition, and the acid and second acid combined is about 3% of the composition, by weight.

10. The composition of claim 9, wherein the acid and second acid is sodium acid pyrophosphate and sodium bisulfate wherein the sodium acid pyrophosphate is about 2% of the composition and the sodium bisulfate is about 1% of the composition.

11. The composition of claim 1, diluted between about 10-fold to about 200-fold in water.

12. The composition of claim 10, diluted between about 10-fold to about 200-fold in water.

13. The composition of claim 10, diluted about 25- to 30-fold in water.

14. A method for treating a foot of an animal, the method comprising contacting the foot of the animal with the composition of claim 11.

15. The method of claim 14, wherein the foot of the animal is contacted with the composition of claim 13.

16. The method of claim 14, wherein the animal is an ungulate.

17. The method of claim 16, wherein the ungulate is a dairy cow.

18. The method of claim 14, wherein contacting comprises bathing, spraying or dipping.

19. The method of claim 14, wherein the animal has, or is at risk for, an infectious disease of the foot.

20. The method of claim 19, wherein the infectious disease of the foot is hairy heel warts, foot rot, or foot scald.
